(19) United States
(12) Patent Application Publication
(10) Pub. No.: US 2008/0039830 A1
(43) Pub. Date: Feb. 14, 2008

(54) Method and Apparatus for Ablative Recanalization of Blocked Vasculature

(76) Inventors: Gareth T. Munger, St. Louis, MO (US); Ashwini K. Pandey, St. Louis, MO (US); Raju R. Viswanathan, St. Louis, MO (US)

Correspondence Address: Bryan K. Wheelock, Suite 400, 7700 Bonhomme, St. Louis, MO, 63105, US

(21) Appl. No.: 11/838,794

(22) Filed: Aug. 14, 2007

Related U.S. Application Data
(60) Provisional application No. 60/837,485, filed on Aug. 14, 2006.

(51) Int. Cl. A61B 18/18

(52) U.S. Cl. 606/33

(57) ABSTRACT

A method of treating vessel occlusions including chronic total occlusions (CTO) of the coronary arteries, and to generally remove tissue material, is presented that relies on remotely actuated navigation of an interventional RF-capable ablation device to the occlusion and controlled application of ablative RF energy. The combined use of remote navigation-based precision control of the distal end of the device and application of ablative energy enables crossing of elongated lesions and CTOs, calcified lesions and CTO's, lesions and CTO's located at vessel branches, and in general the removal of tissue material at a chosen tissue location.

### CROSS-REFERENCE TO RELATED APPLICATION

This application claims priority to prior U.S. Patent Application Ser. No. 60/837,485, filed Aug. 14, 2006, the entire disclosure of which is incorporated herein by reference.

### FIELD

This invention relates to methods, devices and systems for occlusion and chronic total occlusion (CTO) ablation therapy and particularly to the treatment of occlusive coronary artery lesions.

### BACKGROUND

Interventional medicine is the collection of medical procedures in which access to the site of treatment is made by navigation through one of the subject's blood vessels, body cavities or lumens. Interventional medicine technologies have been applied to the manipulation of medical instruments such as guide wires and catheters which contact tissues during surgical navigation procedures, making these procedures more precise, repeatable, and less dependent on the device manipulation skills of the physician. Remote navigation of medical devices is a recent technology that has the potential to provide major improvements to minimally invasive medical procedures. Several presently available interventional medical systems for directing the distal end of a medical device use computer-assisted navigation and a display means for providing an image of the medical device within the anatomy. Such systems can display a projection or cross-section image of the medical device being navigated to a target location obtained from an imaging system such as x-ray fluoroscopy or computed tomography; the surgical navigation being effected through means such as remote control of the orientation of the device distal end and proximal advance of the medical device.

In a typical minimally invasive intervention data are collected from a catheter or other interventional device instrumentation that are of significant use in treatment planning, guidance, monitoring, and control. For example, in diagnostic applications right-heart catheterization enables pressure and oxygen saturation measure in the right heart chambers, and helps in the diagnosis of valve abnormalities; left-heart catheterization enables evaluation of mitral and aortic valvular defects and myocardial disease. In electrophysiology applications, electrical signal measurements may be taken at a number of points within the cardiac cavities to map cardiac activity and determine the source of arrhythmias, fibrillations, and other disorders of the cardiac rhythm. For angioplasty applications a number of interventional tools have been developed that are suitable for the treatment of vessel occlusions: guide wires and interventional wires may be proximally advanced and rotated to perform surgical removal of the inner layer of an artery when thickened and atheromatous or occluded by intimal plaque (endarterectomy). Reliable systems have evolved for establishing arterial access, controlling bleeding, and maneuvering catheters and catheter-based devices through the arterial tree to the treatment site. Systems for coronary arteries are similar, but the smaller size (3 to 5 mm proximally) and greater tortuosity of the coronaries require smaller and more flexible devices.

The primary objective of angioplasty is to re-establish a stable lumen with a diameter similar to that of the normal artery. This goal may be achieved by using a variety of interventional devices, including angioplasty balloons, lasers, rotoblators, and stents. In recent years the introduction of specially designed catheters comprising strong inflatable balloons at or near their distal end, as well as along the length of the device, has greatly changed the field of minimally invasive cardiovascular surgery. The balloons are used for percutaneous transluminal coronary angioplasty (PTCA) to dilate a partially obstructed artery and restore blood flow to the myocardium; balloons catheters are also used to treat heart valve stenosis. Although there are risks associated with the procedure, such as tearing or embolization, the technique may be applied to several coronary arteries with excellent results, and may be repeated if necessary. All new developments in the field of percutaneous coronary intervention (PCI) have been targeted to do one or more of the following: i) reduce treatment risk; ii) reduce the occurrence of restenosis; and iii) allow more complex cases to be treated via minimally invasive techniques. In particular, a number of new devices and associated techniques have been developed in an attempt to increase the chronic total occlusion (CTO) treatment success rate; up to now however the use of devices to increase the success rate in angioplasty of CTO has been accompanied by an increase in complication rate.

Restenosis is the major limitation of angioplasty. Restenosis is a complex process comprising three separate mechanisms: early recoil, neo-intimal hyperplasia, and late contraction (negative remodeling). Arterial plaque begins in the intima by deposits of fatty debris from blood. As the disease progresses lipids accumulate in the intima to form yellow fatty streaks. A fibrous plaque begins to form. Eventually a complex lesion develops as the core of the fibrous atherosclerotic plaque necroses, calcifies, and hemorrhages. Angioplasty leads to a fracture of the atherosclerotic plaque, the intima, and sometimes fractures extending into the media. Immediately following balloon angioplasty, the elastic medial vessel layer contracts (early recoil). Over weeks, neo-intimal cell proliferation results in new tissue growth occupying the cracks and tears in the vessel wall, new tissue becomes less cellular and the healing sites begins to resemble a fibrous plaque (neo-intimal hyperplasia). In most patients the lumen enlarging effect of angioplasty outweighs the lumen-narrowing effect of neo-intimal hyperplasia. However in about 40% of patients neo-intimal hyperplasia is excessive and results in clinically symptomatic restenosis within three to six months. This effect is compounded by late arterial contraction (negative remodeling).

Angioplasty enlarges the lumen by stretching and splitting the wall; in some cases this is made impossible by lesions with a lumen too small for the balloon to cross, or by heavy calcification of the arterial wall, making it too tough and inelastic to split or stretch. In these cases it may be necessary to remove tissue by cutting (atherectomy device), abrading (rotoblator), or vaporizing (laser). Because the risk of arterial wall perforation is clearly much higher with these methods, they are usually not applied aggressively to achieve the desired final lumen size; rather, they are used to initially “debulk” the lesion, and then followed by balloon angioplasty and/or stent placement.

Stent placement following angioplasty effectively repairs vessel wall dissections, prevents tissue flaps from protruding in the lumen, resists elastic recoil, and minimizes loss of lumen diameter due to negative remodeling. Stents by themselves however do not eliminate restenosis as they appear to stimulate proliferation. Restenosis is best addressed by placing a drug eluting stent in the balloon-treated lesion or by irradiating the treated vessel segment by brachytherapy. These restenosis preventive treatments have made a profound impact on the mid and long-term viability of narrow vessel and CTO disease treatment.

Chronic total occlusions are present in about 30% of the 1.5 million diagnostic angiograms performed every year in the United States. However, up to now minimally invasive treatment of CTOs has been difficult, and only about 10% of angioplasty interventions are directed at CTO therapy; indeed CTO presence often precludes treatment by coronary percutaneous intervention and remains a major reason for referral for coronary artery bypass graft surgery (CABG). Treatment success rate is typically in the 60%-85% range; yet a significant number of CTO lesions are left untreated because of uncertainties regarding procedural success and long term benefit. Procedural shortcomings and complications include failure to cross with the guide wire or balloon, failure to dilate the lesion, failure to deploy a stent, and myocardial infarction. Additional risks include distal perforation and/or arterial dissection and associated complications such as haemo-pericardium, cardiac tamponade, and death, and the possible need for prompt pericardiocentesis and reversal of anticoagulation and/or emergency CABG surgery; and embolization. In general, attempts at treating CTOs with current technologies are not recommended when: i) the CTO presents an extended blockage, for example greater than 15 mm; ii) the CTO is heavily calcified; iii) there is poor distal vessel visualization, and the introduction of a retrograde wire is difficult or there is no prospect for retrograde access; iv) the CTO has been present for an extended period of time, for example more than three months; v) the lesion presents with irregular contours, in eccentric anatomy, or with antegrade collaterals; or vi) thrombus is present. However recent clinical data indicate that successful CTO treatment and artery opening induce significant long-term morbidity and mortality advantages, including reduction or elimination of angina pectoris symptoms, improved left ventricular function and ejection fraction, reduced myocardial infarction and lower incidence of cardiac death. Clinical data support aggressive attempts to open chronically occluded vessels when favorable treatment factors exist such as the presence of a tapered stump at a branch, pre- or post-branch occi, absence of bridging collateral vessels, and presence of a functional occlusion. New techniques capable of safely and effectively treat the most difficult cases would most likely induce significantly favorable clinical outcomes.

New CTO techniques developed recently include mechanical and ablative approaches. Mechanical technologies include the use of polymer coated or tapered wires, low profile balloons, blunt micro-dissection to attempt to gently separate atherosclerotic plaques in various tissue planes to create a passage through the CTO by using the elastic properties of adventitia versus the inelastic properties of fibro-calcific plaque to create fracture planes. Ablative technologies include the use of excimer lasers, ultrasound or vibrational techniques (activated guide wire angioplasty) to induce cavitation, as for example by delivering controlled acoustic energy along the active section of a thin wire; the infusion of collagenase at the CTO through a thin catheter to soften the occlusion and enable wire crossing; and the recent development of radio-frequency (RF) approaches. Stent deployment, if the artery can be opened, has been shown to improve outcome. In particular balloon angioplasty data indicate that the need for emergency CABG has fallen since stenting has become routine. Stiff guide wires, while providing increased pushability and torque response are more likely to create false channels, dissection and perforation. Hydrophilic guide wires have a polymer coating that becomes very slippery once moistened, which reduces thrombus adhesion and facilitates the advancement of the wire within the occlusion.

Bifurcation CTO lesions in small vessels are particularly difficult to treat. Identification of the best approach to bifurcation disease remains unresolved. It is debatable whether PCI using current technology is the treatment of choice for such cases because of technical problems and high incidence of acute and chronic events.

An excimer laser wire was developed to attempt crossing CTOs in the event of a failure with any guide wire. As the results of the TOTAL trial (Total occlusion trial with angioplasty by using laser guide wire) indicate, although laser guide wire technology was safe, the increase in crossing success did not reach statistical significance. The most frequent reasons for laser guide wire failure were false route tracking and misalignment, while the most common reason for failure in the mechanical wire group was absence of wire progression. Accordingly, increasing lesion penetration power by itself is not sufficient to lead to significant favorable clinical outcomes.

U.S. Pat. No. 6,394,956 issued to Chandrasekaran et al, and assigned to Scimed Life Systems, Inc., (incorporated herein by reference) discloses a combination catheter including an intravascular ultrasound (IVUS) device and an RF ablation electrode. RF ablation proceeds by depositing energy to locally raise the tissue temperature to fulguration. RF power for inter-arterial lesion ablation is typically delivered in pulses to allow heat dissipation and avoid damaging adjacent healthy tissues. In one embodiment pulses are delivered at a rate of about 10 Hz to about 10 kHz. Each ablative pulse is typically delivered with a frequency of about 200 kHz to about 2 MHz, although a typical electrosurgical power generator might operate within a frequency range from about 200 kHz to about 35 MHz. The RF circuit voltage may be as high as 1 kV, and delivered power in the range 1 to 50 watts depending on the application. Ultrasound imaging provides feedback regarding the relative position of the device distal end and vessel tissues, so as to reduce the risks associated with RF energy delivery to the vessel walls. Various RF electrode configurations are possible, including protruding hemispherical shape, roughened protruding hemispherical, concave electrode surface, or extendable intermeshed wires enabling variable electrode diameter. U.S. Pat. No. 6,394,956 describes a mechanical system of pull-wires for manually operated navigation, but does not address its limitations, including limitations on fine control of distal end steering Further, Intravascular Ultrasound remains a niche product with mostly research applications despite its potential value in visualizing true lumen dimensions.

Other recently developed techniques include the use of optical coherence reflectometry (OCR) for the characterization of tissues. OCR uses an optic fiber placed through a support catheter or guide wire to illuminate tissue with a low coherence light; reflected and scattered light patterns are detected and analyzed to differentiate between plaque and normal arterial wall; it has been shown that light scattering intensity increases when scattering originates from a healthy arterial wall as compared to arterial occlusive materials. U.S. Pat. No. 6,852,109 issued to Winston and Neet and assigned to Intraluminal Therapeutics, Inc., (incorporated herein by reference) describes a guide wire assembly including a guide wire electrically connected to an RF power generator and an optical fiber connected to an optical reflectometer. The assembly may comprise either a unipolar or bipolar RF electrode(s). RF power may be gated to an ECG signal to ensure that power is not delivered during the ECG S-T segment, as the heart is most sensitive to electrical signals during this period. Also, RF sub-system design may include a control to ensure that RF power is delivered only when the RF electrode is in tissue contact. Although combination of RF ablation capability with OCR characterization helps to reduce adverse events, such as arterial perforation or dissection, the methods and devices disclosed in U.S. Pat. No. 6,852,109 do not teach nor suggest how to improve on the state-of-the-art for device distal end navigation, localization, and fine adjustment of local positioning with respect to the vessel walls and lesions. In clinical trials utilizing the technology described in U.S. Pat. No. 6,852,109 limited steerability (in particular within the lesions) remained a problem.

The present invention addresses the need for fine, precise control of distal tip steering and maintenance of device tip alignment with the longitudinal vessel direction. It also describes methods to increase the efficiency of power delivery and make the ablation process more effective, while at the same time avoiding unduly large temperature increases, and methods of coordinating power delivery with tip position and steering control. It also provides a method for creating and enlarging a pathway through a blocked blood vessel with partial or total blockage.

### SUMMARY

Three technology requirements for the crossing of most challenging CTOs are addressed by embodiments of the present invention: increased lesion penetration power as compared to guide wires without the need for large proximal force application; tissue characterization and differentiation capability to reduce the likelihood of adverse events; and steerability of the device distal end to keep the ablation device oriented along the main local vessel axis, therefore enabling ablative power application. Embodiments of the present invention provide a method of performing CTO ablation therapy by guiding a wire, catheter or interventional device to the occlusion, possibly characterizing the tissues in the vicinity of the device distal end, orienting an RE ablation electrode, applying RF power to the occlusion through the wire or catheter, and iteratively navigating the wire or catheter through the lesion with or without local tissue characterization, and applying RF power to create an opening therethrough. The invention discloses methods of delivering power to the lesion in an effective manner, and the coordination of tip position control and power delivery. Further, embodiments of the invention also provide a method of navigating an RF-capable therapy device by magnetic navigation means, mechanical navigation means, electrostrictive navigation means, and combination thereof. Use of magnetic navigation in combination with RF ablation enables the use of thinner, more maneuverable wires as pushability requirements decrease. Current CTO intervention failures stem from either inability to cross the occlusion with a guide wire, or from lesion restenosis or reocclusion. Restenosis is a particularly significant problem for small (<3 mm) vessel disease. The ability to cross the lesion with a thinner wire enables advancement of a lower profile balloon catheter, and thus the treatment of smaller arteries including the capability of placing stents and drug-eluting stents (or the use of brachytherapy) in smaller arteries. Stents address both elastic vessel recoil and negative remodeling; drug eluting stents have a robust effect on tissue growth and very significantly bring down the rate of restenosis. Accordingly both CTO treatment failure modes are addressed by magnetic navigation of an RE ablation device as described below.

### BRIEF DESCRIPTION OF THE DRAWINGS

FIG. 1-A shows a patient positioned in a projection imaging system for an interventional procedure such as percutaneous coronary intervention (PCI) and therapy using a controlled minimally invasive modality, such as RE ablation;

FIG. 1-B illustrates an interventional device distal end being in occlusion contact within a theater of intervention such as an artery;

FIG. 2 presents a workflow chart for a method of coronary intervention and chronic total occlusion therapy according to the present invention;

FIG. 3 schematically shows a radio-frequency interventional device creating a crossing through a vessel CTO, and

FIG. 4 schematically illustrates the use of an interventional device according to the principles of the present invention for the treatment of a CTO at a vessel branch.

Corresponding reference numerals indicate corresponding points throughout the several views of the drawings.

### DETAILED DESCRIPTION

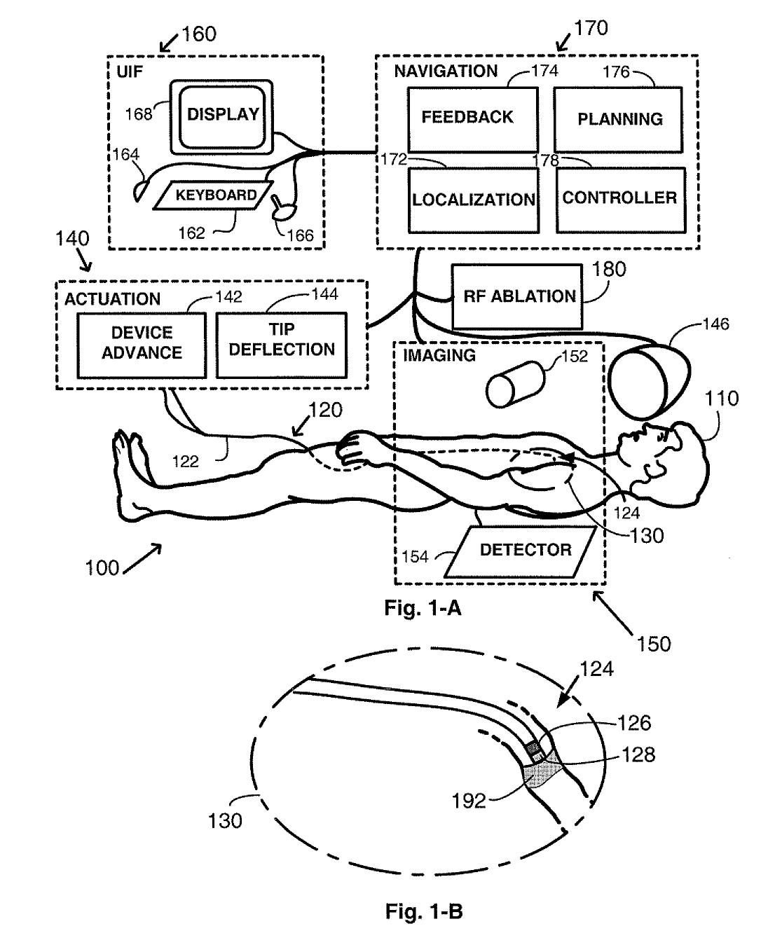

As illustrated in FIG. 1, a patient 110 is positioned within an interventional system, 100. An elongated navigable medical device 120 having a proximal end 122 and a distal end 124 is provided for use in the interventional system 100, FIG. 1-A, and the medical device is inserted into a blood vessel of the patient and navigated to an intervention volume 130. A remote navigation or remote actuation means of applying force or torque to orient the device distal end 124 is provided, as illustrated by actuation block 140 comprising a device advance/retraction component 142 and a tip deflection component 144. The tip deflection means may be one of (i) a mechanical pull-wire system; (ii) a hydraulic or pneumatic system; (iii) an electrostrictive system; (iv) a magnetic system; or (v) other navigation system as known in the art. For illustration with a preferred embodiment, in magnetic navigation a magnetic field externally generated by a magnet(s) assembly 146 orients a small magnet located at the device distal end (126, FIG. 1-B).

Real time information is provided to the physician by an imaging sub-system 150, for example an x-ray imaging chain comprising an x-ray tube 152 and an x-ray detector 154, and also possibly by use of a three-dimensional device localization sub-system such as a set of electromagnetic wave receivers located at the device distal end (not shown) and associated external electromagnetic wave emitters (not shown), or other localization device with similar effect such as an electric field-based localization system that is based on sensing an externally applied voltage gradient. In the latter case the conducting body of the wire itself carries the signal recorded by the tip electrode to a proximally located localization system.

The physician provides inputs to the navigation system through a user interface (UIF) sub-system 160 comprising user interfaces devices such as a display 168, a keyboard 162, mouse 164, joystick 166, and similar input devices. Display 168 also shows real-time image information acquired by the imaging system 150 and the three-dimensional localization system. UIF sub-system 160 relays inputs from the user to a navigation sub-system 170 comprising a 3D localization block 172, a feedback block 174, a planning block 176, and a controller 178. Navigation sequences are determined by the planning block 176 based on inputs from the user, possibly pre-operative data and localization data processed by localization block 172, and real-time imaging and feedback data processed by feedback block 174; the navigation sequence instructions are then sent to the controller 178 which actuates the device through actuation block 140 to effect device advance and tip deflection. Other navigation sensors might include an ultrasound device or other device appropriate for the determination of distance from the device tip to the tissues or for tissue characterization (not shown). Further device tip feedback data may include relative tip and tissues positions information provided by a local imaging system, predictive device modeling, or device localization system. In the application to occlusion ablation, additional feedback may be provided by an IVUS device, an optical coherence reflectometry device, or similar device that allows intravascular and vascular characterization to separate plaque or fibrous lesion from vascular wall (not shown).

In closed loop implementation, the navigation sub-system 170 automatically provides input commands to the device advance and tip orientation actuation components based on feedback data and previously provided input instructions; in semi-closed loop implementations, the physician fine-tunes the navigation control, based in part upon displayed and other feedback data, such as haptic force feedback information. Control commands and feedback data may be communicated from the user interface 160 and navigation sub-system 170 to the device and from the device back to navigation sub-system 170 (feedback) through cables or other means, such as wireless communications and interfaces. As known in the art, system 100 comprises an electromechanical device advancer 142, capable of precise device advance and retraction based on corresponding control commands. In RF therapy applications, in one preferred embodiment an RF component 180 may collect temperature data measured at the device tip 124 by electrode 128 in contact with tissue, FIG. 1-B.

The RF-capable device is advanced into contact with the occlusion 192 and positioned such that its tip orientation is aligned with the local vessel tangent direction. In a preferred embodiment, the vessel centerline information is available to the navigation system, either from user marking of contrast-filled vessel lumen from two angularly separated X-ray images, or from an image processing-based extraction of the three dimensional vessel contour from two or more angularly separated X-ray images. In some cases this centerline information can also be extracted by either automated or semi-automated means from a three dimensional preoperative or intraoperative image such as a CT scan. In cases where the vessel is completely occluded, local image information may not be available. In such cases the navigation system may offer a means of interpolating vessel centerline geometry, based possibly on user definition of a putative centerline. The vessel centerline information is used by the navigation system to suitably actuate the device tip in order to maintain a tip orientation that is substantially aligned with the local tangent to the vessel centerline. For example, in the case of a magnetic navigation system, a suitably oriented magnetic field is applied that causes the magnetically endowed device tip to approximately align with the local vessel centerline tangent. The applied magnetic field may in some cases be defined with an oversteer included to account for restoring forces due to device elasticity. In a preferred embodiment a computational device model can be used together with vessel geometry to compute a suitable amount of field direction oversteer to be applied.

Once the device tip is suitably aligned with the local vessel tangent, RF power is applied, and the device is navigated through the occlusion by advancing it through a restricted or small amount. The opening thus created by the device or wire tip can be further enlarged by employing the following method: (i) the magnetic field direction is oriented by a restricted, possibly user-defined angular amount away from the field direction B₀ which yields alignment with the vessel centerline; (ii) the field direction is set to precess about B₀; RF ablation energy is applied while this precession is in effect, thus, creating an approximately circular cut in the vessel occlusion. In one embodiment a sequence of such cones with increasing cone angles can provide a suitably large opening up of the vessel occlusion. In an alternate preferred embodiment a different geometrical pattern such as a spiral movement of the magnetic field about B₀ could be employed to enlarge the opening. It is worth noting that the examples here are provided for illustration only and alternate geometric patterns or schemes of movement can be devised by those skilled in the art. During the movement process, RF power can be applied continuously or in pulses, and the power delivery can be performed in any of a variety of pre-defined sequences.

Once the blockage is locally opened up through this “coring” operation, the device is further advanced a little if possible and centered again to locally align with the vessel. Iteration of the above sequence, under real-time imaging, and possibly including local tissue characterization, and/or temperature and/or localization control, enables crossing the CTO. RF electrode design depends on a number of parameters, such as target vessel size, expected occlusive materials to be ablated and other parameters as known in the art.

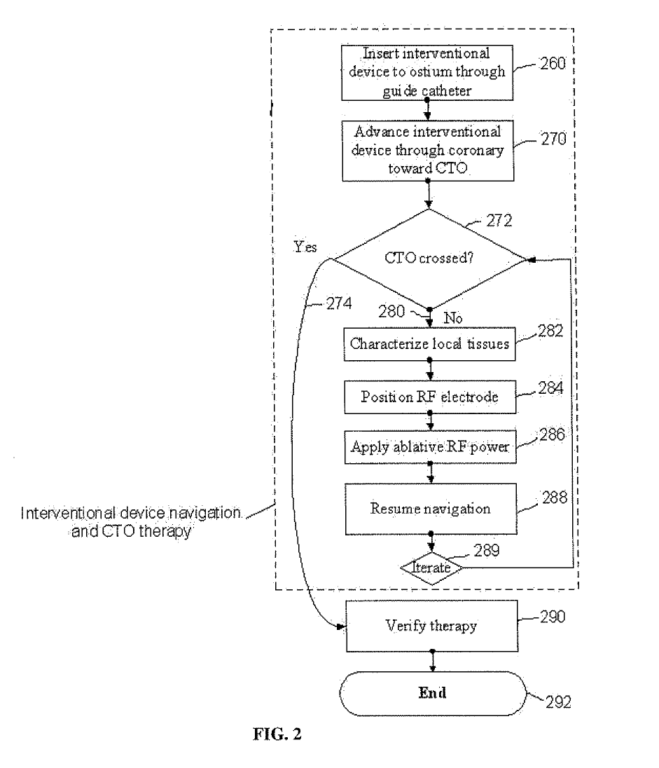

Referring now to FIG. 2, a flow-chart for one embodiment of a method of CTO ablation therapy according to the principles of the present invention is presented, as applied to the treatment of a coronary artery occlusion with interventional device magnetic navigation. A guide catheter for the interventional guidewire or device is inserted into a suitable vessel ostium, for example the entry into the Left Main Artery, in step 210. The interventional device is passed through the guide catheter in order to be navigated to the lesion of interest. In a preferred embodiment, the interventional device is a magnetic guidewire made of an electrically conducting material and with at least one magnetic element in its distal region. The distal tip of the device includes an electrode portion that can deliver RF energy to tissue it is in contact with. The guidewire includes an outer layer of electrical insulation along its entire length up to the proximal portion of the exposed tip electrode. The guidewire is navigated to the proximal portion of the occluded vessel, possibly with magnetic actuation to suitably orient the device tip at various positions along the vessel, as in step 270. At decision block 272, if the CTO was crossed by advancing the interventional device, 274, the coronary blood flow and pressures may be measured or other steps taken, to verify the therapy, 290.

Otherwise, step 280, local tissues in the vicinity of the RF electrode can characterized in one embodiment for example by use of IVUS or OCR, 282. The device distal end and RF electrode are positioned in contact with the lesion and oriented with respect to the local vessel and occlusion anatomy to ensure lesion ablation while respecting the integrity of the arterial wall, 284, ablative RF power is applied (possibly under temperature and localization control), 286, the interventional device is navigated through the lesion opening just created, 288, and the method is iterated 289 till the CTO is crossed, 274.

The application of RF power can take one of a number of different delivery profiles. The frequency used can range from 100 KHz to about 5 MHz. In one preferred embodiment, the RE generator used to produce the RF power can have a frequency in the range of about 450-520 KHz, while in another preferred embodiment it can have a frequency in the range of about 3.8-5 MHz. In one preferred embodiment the RF power can be a steady sinusoidal, square wave, or other periodic waveform applied for a certain time interval, while in another preferred embodiment it can be pulsed with pulses of duration T1 repeated over time intervals T2. The voltage applied can be as high as 1100 V, while more preferably it can be in the range of 10-500 V. The applied current can be as high as 1.5 A, while more preferably it can be in the range 0-500 mA. The power associated with the generated RF energy can be as high as 50 W. Generally the desired power level can be set on the generator. In some specific applications such as CTO recanalization, 25 W may be a useful power setting for the generator. The pulse duration T1 can range from about 0.1 μs to about 5 s, while the repetition time T2 can range from about 20 μs to about 1 s.

In one embodiment of the invention, the power delivery is coordinated with the remote positioning of the device near the target area. The RF generator communicates with the remote navigation system through a communication interface so that the navigation system has the real-time power delivery profile information available to it. This information can be used by the remote navigation system to determine a device actuation profile that is coordinated with the power delivery. For instance, in one embodiment of this invention, in a blocked vessel that is locally curved, it may be necessary to steer or bend the device progressively as the device is advanced in order to conform to the vessel geometry and to ensure that the device stays inside the boundary defined by the wall of the vessel. RF power delivery with simultaneous steering (for instance, changing the orientation of an applied magnetic field in the case of a magnetic navigation system) can cause the device to bend, “cutting” its way through the blockage as it is actuated. In some cases the device may need to be advanced in conjunction with power delivery while RF power is being delivered, in order to advance the device into the occlusion. Thus simultaneous device actuation and RF power delivery can aid in the clinical application. The communication interface provides a mechanism for ensuring seamless coordination. Pulsed delivery of RF power can be useful in this situation to avoid excessive temperature increases in the distal region of the device and in surrounding tissues. The specific RF generator settings used in this coordinated mode of operation of the remote navigation system and RF generator can lie within the ranges identified above. The coordination of the systems can be implemented in different ways. In one preferred embodiment, the device is advanced by a small amount with every RF pulse applied; in this case for example the repetition times between applications could be 0.2 s or larger. In one continuous mode of coordinated operation the RF power pulses can be continuously applied with defined T1 and T2 values, while the device is being advanced at a steady rate. In one embodiment the distance advanced between pulses can take a value close to the length of tissue ablated away in front of the device for every applied RF pulse. In one mode of operation the angular change in orientation of the device can be made to occur at a rate that is dependent on the rate of RF pulse application, (1/T2).

In an alternate embodiment the device actuation or advancement can be controlled manually while RF power is being delivered. For example this may be a preferred method in the absence of a communication interface. The RF generator can produce an audible noise or flashing light or other indication to indicate that power delivery is actively in progress, while the physician manually operates the placement of the device.

As stated above, the pathway through the occlusion can be enlarged as desired by making suitably restricted patterned magnetic field adjustments in conjunction with further ablation. Finally the therapy is verified in step 290 and the method terminates 292. Alternatively to IVUS or OCR, other methods such as optical coherence tomography may be used, as known in the art.

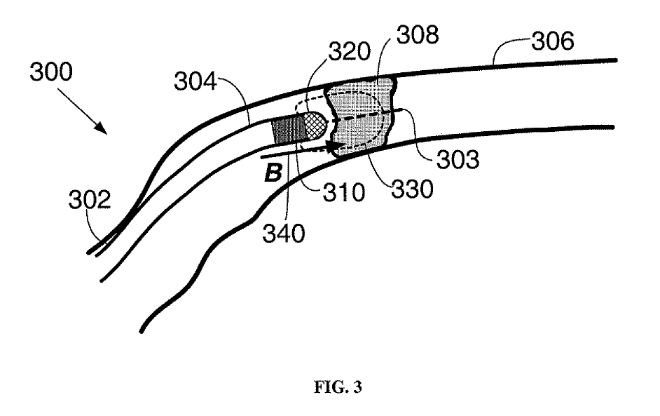

FIG. 3 schematically presents 300 a magnetically navigated RF interventional device 302 being navigated through an artery 306 to contact a CTO occlusion 308. The distal end 304 of the device comprises a magnet 310 sufficient for magnetic navigation in an applied field of about 0.1 Tesla, and preferably no more than about 0.08 Tesla, and preferably no more than about 0.06 Tesla. The device tip comprises an RF electrode 320 for application of ablative power to a lesion volume 330. During the intervention, a magnetic field B 340 externally generated by sub-system 146 is applied to align the device distal end 304 with the local vessel axis 303; pressure is exerted to the lesion by proximally controlling the device advance and RF power is applied, typically in a sequence of pulses. In one preferred embodiment the advancement of the wire is controlled remotely by the physician operating a user input interface such as a joystick, while the wire itself is advanced mechanically by an advancer unit controlled by the user input interface. In another preferred embodiment the advancement of the wire can be controlled directly in automated fashion by the navigation system. It is possible to even integrate control of the RF power delivery system with the navigation system, so that small, precise movements can be suitably coordinated with ablative power delivery for optimal path creation.

RF power delivery can cause high temperatures to be reached locally at the tip of the wire in the distal electrode region. In one embodiment of a magnetic guidewire that is used for RF power delivery, the magnetic material in the guidewire is accordingly a hard magnetic material with high coercivity and suitably high remnant magnetization as well as a suitably high Curie temperature, so that the heating of the tip upon RF power delivery does not result in a large magnetization loss. Examples of such materials are Neodymium-Iron-Boron, Samarium-Cobalt ceramic magnets, suitably heat-treated Platinum-Cobalt alloys, etc. In a preferred embodiment, the magnet material in the distal portion of the wire is separated from the distal electrode by a small thermally insulating spacer that acts as a temperature shield. In a preferred embodiment, the magnet material is characterized by a remnant magnetization of at least 0.6 Tesla, and possesses a Curie temperature of at least 300° C. The distal electrode itself can range from about 0.5 mm to 4 mm in length, while the spacer can be between 0.1 mm and 4 mm long. The electrode can be made out of an electrically conducting hard magnetic material such as Platinum-Cobalt alloy, or it can be a metal or metal alloy. The spacer can be made out of a polymeric material or other poor thermal conductors known to those skilled in the art. More than one magnetic element can be disposed in the distal portion of the wire and enclosed by the insulating sleeve on the wire described earlier.

Various RF electrode designs for CTO therapy are possible, including a mono-polar design wherein RF power is returned to the RF generator through a patch electrode applied to the patient's skin, the electrode patch typically being positioned on the patient's back. The volume 330 through which a given amount of power is deposited in the lesion is dependent upon RF electrode design parameters and local tissue characteristics, as known in the art. Iterative application of ablative power and device navigation under real-time temperature, localization and imaging control enables crossing most CTOs. In particular, use of RF ablative power enables treatment of elongated CTOs as well as crossing densely calcified lesions. It is emphasized that by design of the interventional system and device, maneuverability of the device distal end in most cases enables positioning and orientation of the RF electrode such that only diseased tissue at a safe distance margin from the vessel wall are ablated.

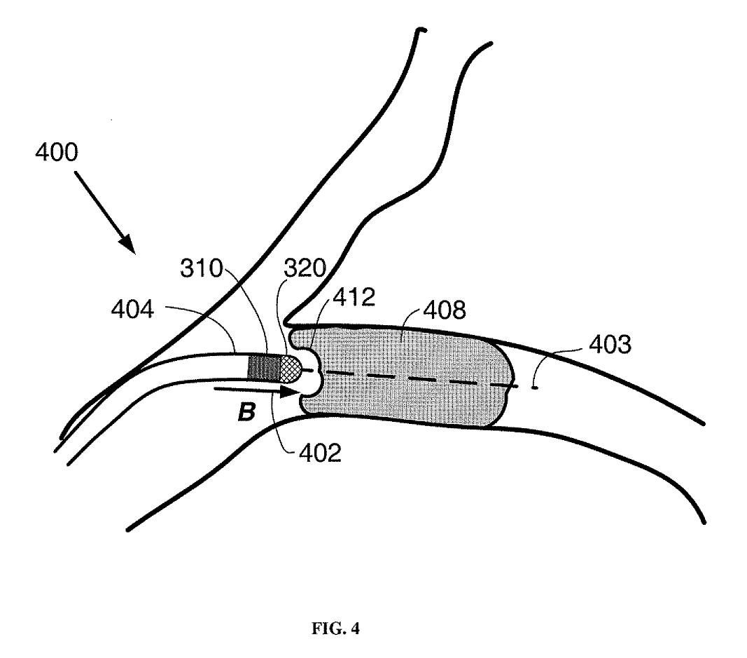

Referring now to FIG. 4, the method of the present invention is applied to the treatment of a branch CTO, 400. Branch CTOs are among the most difficult cases of narrow artery disease to treat with current state-of-the-art technologies. The relative length of the lesion (as for example longer than 15 mm) makes it very unlikely to be successfully crossed by conventional approaches using thin tapered mechanical guide wires. When attempting CTO crossing by advancing a thin tapered wire, the geometry of the vessels and the presence of a lesion at a vessel branch often lead to device prolapse into the adjacent vessel. Alternatively presence of the lesion at the branch without a tapered stump would likely lead to distal wire sliding into the adjacent, non-occluded, branch, and failure to perform therapy. When using magnetic navigation, an externally generated B field 402 is applied to the device distal end 404 comprising a small magnet 310, to align the device with the local vessel axis 403. RE power is applied to electrode 320 when the device tip is in contact with the lesion 408 at surface 412. Iterative application of ablative power and magnetic navigation and device advance enables lesion ablation along the local vessel axis 403 and successful CTO crossing. The use of ablative RF power in combination with magnetic navigation enables creation of a passage way through the lesion with minimum proximal advance force being applied, thereby avoiding distal device buckling and prolapse, and avoiding distal end slippage away from the lesion and into the patent branch.

When a pathway through the occlusion is thus opened, it is followed by delivery of a balloon angioplasty catheter, stent delivery catheter or other therapy delivery device. Such a device can closely follow the RF wire in order to aid in further opening the pathway to cross the lesion for therapy delivery.

Although the method has been illustrated for magnetic navigation applications, it is clear that it may also be applied in conjunction with other means of navigation. For example, the navigation means may comprise mechanical actuation, as per use of a set of pull-wires that enable distal device bending, by itself or in conjunction with proximal device advance and rotation. The navigation means may also comprise other techniques known in the art, such as electrostrictive device control. Further navigation means may comprise combination of the above methods, such as combination of magnetic and electrostrictive navigation, combination of mechanical and electrostrictive navigation, or combination of magnetic and mechanical navigation.

The advantages of the above described embodiments and improvements should be readily apparent to one skilled in the art, as to enabling CTO and occlusive lesion ablative therapy. Additional design considerations may be incorporated without departing from the spirit and scope of the invention. Accordingly, it is not intended that the invention be limited by the particular embodiment or form described above, but by the appended claims.

1.-17. (canceled)

18. A system for recanalization of blocked vasculature, comprising:
i. a remote navigation system for remote actuation of an interventional device within a patient's lumen to an occlusion;
ii. a remotely actuated interventional device capable of Radio Frequency power delivery to tissue;
iii. remote actuation means for orienting the interventional device distal end with respect to the occlusion in the vasculature;
iv. means for applying Radio Frequency energy to the occlusion in order to create a pathway through the occlusion; and
v. means to expand the radial dimensions of the pathway thus created.

19. The system of claim 18, where the system further includes a means of local characterization of tissue in the occluded vessel.

20. The system of claim 18, where the remote navigation system is a magnetic navigation system.

21. The system of claim 20, where the interventional device includes magnetic material that responds to remote actuation and which is characterized by (i) a remnant magnetization of at least 0.6 Tesla, and (ii) a Curie temperature of at least 300° C.

22. The interventional device of claim 21, where the device includes an electrode at the distal tip for Radio Frequency power delivery that is separated from the magnetic material by a spacer element between 0.1 mm and 4 mm in length.

23. The interventional device of claim 21, where the magnetic material is also an electrode capable of Radio Frequency power delivery.

24. (canceled)

25. (canceled)

26. (canceled)

27. (canceled)

28. A method of performing tissue ablation to remove tissue material in a subject body, comprising:
(i) magnetically navigating the distal end of a magnetically endowed interventional device, capable of Radio Frequency (RF) power delivery, to a tissue location;
(ii) orienting the distal end of the interventional device;
(iii) applying ablative RF energy to the tissue location in pulsed form while navigating the interventional device; and
(iv) iterating through steps i) to iii) to remove tissue material.

29. The method of claim 28, where the RF energy is applied in a frequency range of between about 300 KHz and about 800 KHz.

30. The method of claim 28, where the RF energy is applied in a frequency range of between about 2 MHz and about 6 MHz.

31. The method of claim 28, where the RF energy is applied with a voltage of less than about 1100 V.

32. The method of claim 28, where the current corresponding to the RF energy delivery is less than about 1.5 A.

33. The method of claim 28, where the RF pulses are applied with a pulse duration between about 0.1 μs and about 5 seconds.

34. The method of claim 28, where the repetition time between one RF pulse and an immediately successive one is between about 20 μs and about 1 second.

35. A system for performing tissue ablation to remove tissue material in a subject body, comprising:
(i) a remote navigation system for remotely navigating the distal end of an interventional device endowed with remote actuation means and capable of Radio Frequency (RF) power delivery, to a tissue location; and
(ii) a Radio Frequency generator connected to the interventional device and capable of delivering pulsed Radio Frequency energy through said device to the tissue location.

36. The system of claim 35, where the RF energy is applied in a frequency range of between about 300 KHz and about 800 KHz.

37. The system of claim 35, where the RF energy is applied in a frequency range of between about 2 MHz and about 6 MHz.

38. The system of claim 35, where the RF energy is applied with a voltage of less than about 1100 V.

39. The system of claim 35, where the current corresponding to the RF energy delivery is less than about 1.5 A.

40. The system of claim 35, where the RF pulses are applied with a pulse duration between about 0.1 μs and about 5 seconds.

41. The system of claim 35, where the repetition time between one RF pulse and an immediately successive one is between about 20 μs and about 1 second.
